(19) United States
(12) Patent Application Publication
(10) Pub. No.: US 2013/0150833 A1
(43) Pub. Date: Jun. 13, 2013

(54) SURGICAL INSTRUMENT

(71) Applicants: William J. Peine, Ashland, MA (US); Andres Chamorro, Waltham, MA (US)

(72) Inventors: William J. Peine, Ashland, MA (US); Andres Chamorro, Waltham, MA (US)

(73) Assignee: Cambridge Endoscopic Devices Inc., Framingham, MA (US)

(21) Appl. No.: 13/758,272

(22) Filed: Feb. 4, 2013

Related U.S. Application Data
(63) Continuation of application No. 12/590,452, filed on Nov. 6, 2009.

(51) Int. Cl. A61B 17/00

(52) U.S. Cl. CPC A61B 17/00
USPC 606/1

(57) ABSTRACT

A medical instrument having a proximal control handle and a distal tool that are intercoupled by an elongated instrument shaft that is meant to pass internally of an anatomic body, proximal and distal movable members that respectively intercouple the proximal control handle and the distal tool with the instrument shaft, cable control means disposed between the movable members, an actuation member at the handle for controlling the distal tool through the movable members, a coupler for selectively engaging or disengaging separable cable segments of the actuation member, and the handle housing being provided in separate sections providing a rear opening for receiving the instrument shaft portion.

### TECHNICAL FIELD

The present invention relates in general to medical instruments, and more particularly to manually-operated surgical instruments that are intended for use in minimally invasive surgery or other forms of surgical or medical procedures or techniques. The instrument described herein is primarily for a laparoscopic procedure, however, it is to be understood that the instrument of the present invention can be used for a wide variety of other procedures, including intraluminal procedures.

### BACKGROUND OF THE INVENTION

Endoscopic and laparoscopic instruments currently available in the market are extremely difficult to learn to operate and use, mainly due to a lack of dexterity in their use. For instance, when using a typical laparoscopic instrument during surgery, the orientation of the tool of the instrument is solely dictated by the location of the target and the incision. These instruments generally function with a fulcrum effect using the patient's own incision area as the fulcrum. As a result, common tasks such as suturing, knotting and fine dissection have become challenging to master. Various laparoscopic instruments have been developed over the years to overcome this deficiency, usually by providing an extra articulation often controlled by a separately disposed control member for added control. However, even so these instruments still do not provide enough dexterity to allow the surgeon to perform common tasks such as suturing, particularly at any arbitrarily selected orientation.

The goal of minimally invasive surgery (MIS) is to manipulate tissues within the human body while minimizing damage to the surrounding healthy organs. Laparoscopy, for example, uses endoscopic cameras and long slender instruments to perform surgery through a few small (1-2 cm) skin incisions. This provides many benefits to patients over traditional open incision techniques, including fewer infections, less pain, shorter hospital stays, faster recovery times, and less scaring. These advantages have allowed surgeons to apply MIS to procedures in every surgical specialty. During the 1990's, the growth rate of MIS was tremendous; however, in the last few years the application to new procedures has largely stalled due to limitations in visualization, access, and control. It is a general belief among surgeons that a new wave of technology is needed in order for MIS to reach the next level. Smaller cameras and instruments that can flexibly navigate around organs with added dexterity will allow them to perform surgery not possible today.

Prior laparoscopic and endoscopic instruments were a simple adaptation of tools used for open incision surgery. They are similar in mechanical construction with the addition of a long, 2-10 mm diameter shaft between the handle and end effectors. They lack the dexterity of open incision surgery due to the “fulcrum effect”. Since the tools pivot about the incision, they are generally limited to 5 Degrees-of-Freedom (DOF): pivoting up/down, pivoting left/right, sliding in/out, rotating about the shaft axis, and actuation of the jaws. In contrast, open incision surgery allows full dexterity (7 DOF) due to the surgeon's wrist, with additional DOF from their elbow and shoulder used to avoid obstacles and optimize access to the tissue. Further complicating MIS, the surgeon views the operative site on a monitor located outside the sterile field. This displacement between eyes and hands combined with the reversal of motions caused from the fulcrum effect makes these techniques difficult to learn and master. It takes the skills of an experienced surgeon to consistently perform advanced MIS at a high level.

Surgery now in virtually every surgical discipline is moving toward making MIS more minimal. This means using smaller and fewer incisions, or most ideally, no incisions. The art has already made the transition from open to endoscopic surgery; now surgeons are pioneering surgical techniques that use the patient's natural orifices as entry points into the body. These approaches further reduce pain and recovery times and, in many cases, produce no visible scars. One fairly new technique is referred to as single port access surgery (or SPA). This is a type of laparoscopy where all the instruments and laparoscope enter the abdominal cavity through one incision. Most of these procedures use the umbilicus for the entry port location because it heals quickly, does not have significant muscle groups below it, and hides any scaring well.

An improved instrument is shown in U.S. Pat. No. 7,147,650 having enhanced dexterity and including, inter alia, a rotation feature with proximal and distal bendable members. Other instruments are also shown in U.S. Pat. No. 7,364,582 and U.S. Pat. No. 7,338,513, commonly owned by the assignee of the present invention. Even though these instruments have improved features there remains the need for a more economically feasible instrument and, in particular, an instrument in which the handle can be re-used while the tip of the instrument is disposable or reposable.

Reference is now also made to two other applications co-pending and co-owned with the present invention and identified as published applications 2009/0069842 and 2009/0171147. These applications illustrate various instrument constructions some of which include a disposable or reposable instrument structure.

Accordingly, an object of the present invention is to provide an improved laparoscopic or endoscopic instrument in which a portion of the instrument is re-useable and a portion is disposable or reposable. In embodiments described herein the handle end of the instrument is re-useable and the distal portion or tip of the instrument is disposable or reposable. By being able to re-use the handle portion, the instrument is more economically feasible.

Still another object of the present invention is to provide an improved laparoscopic or endoscopic instrument in which a portion of the instrument is re-useable and a portion is disposable or reposable. By constructing the instrument in this manner this allows the ready substitution of various end effectors useful in performing a surgical procedure.

Still a further object of the present invention is to provide a reposable instrument construction in which the substitutable shaft portion is engaged in a rear entry or rear load manner.

A further object of the present invention is to provide an improved laparoscopic or endoscopic surgical instrument that allows the surgeon to manipulate the tool end of the surgical instrument with greater dexterity.

Another object of the present invention is to provide an improved surgical or medical instrument that has a wide variety of applications, through incisions, through natural body orifices or intraluminally.

Another object of the present invention is to provide a locking feature that is an important adjunct to the other controls of the instrument enabling the surgeon to lock the instrument once in the desired position. This makes it easier for the surgeon to thereafter perform surgical procedures without having to, at the same time, hold the instrument in a particular bent configuration.

Still another object of the present invention is to provide an improved medical instrument that can be effectively controlled with a single hand of the user.

### SUMMARY OF THE INVENTION

To accomplish the foregoing and other advantages and features of the present invention there is provided a surgical instrument comprising: an instrument shaft having proximal and distal ends; a tool disposed from the distal end of the instrument shaft; a control handle coupled from the proximal end of the instrument shaft; a distal motion member for coupling the distal end of the instrument shaft to the distal tool; a proximal motion member for coupling the proximal end of the instrument shaft to the handle; actuation means extending between the distal and proximal motion members for coupling motion of the proximal motion member to the distal motion member for controlling the positioning of the tool; and an actuation cable extending from the handle to the tool for controlling the actuation of the tool. The actuation cable is separated into two inter-engagable cable segments that enable the proximal motion member to be disconnected from the control handle.

In accordance with other aspects of the present invention the surgical instrument may further include a rotation means disposed adjacent the control handle and rotatable relative to the control handle for causing a corresponding rotation of the instrument shaft and tool; at least the proximal motion member may comprise a proximal bendable member, the rotation means may comprise a rotation knob that is adapted to rotate the tool about a distal tool roll axis and the rotation knob may be disposed between the control handle and proximal bendable member; an actuation lever may be supported from the handle at a pivot point on the handle and for controlling the actuation cable; a slider may be provided for capturing the proximal end of said tool actuation cable and an actuation lever supported at the handle for controlling the translation of the slider and, in turn, the operation of the tool; a locking mechanism may be provided for fixing the position of the tool at a selected position and having locked and unlocked states, the locking mechanism including a ball and socket arrangement disposed about the proximal motion member and a cinch member for locking the ball and socket arrangement; the socket member may comprise a split socket and the cinch member closes the split socket to lock the socket on the ball; a horn may be provided that is pivotally supported from the handle and that is operable to engage and disengage the cable segments; and a collet may be supported in the handle for closing about the cable segments.

In accordance with another embodiment of the invention there is provided a medical instrument having a proximal control handle and a distal tool that are intercoupled by an elongated instrument shaft that is meant to pass internally of an anatomic body, proximal and distal movable members that respectively intercouple the proximal control handle and the distal tool with the instrument shaft, cable control means disposed between the movable members, an actuation member at the handle for controlling the distal tool through the movable members, and a coupler for selectively engaging or disengaging separable cable segments of the actuation member.

In related aspects of the present invention the coupler may include a collet attached to one of the cable segments and a capture lug on the other of the cable segments, the collet for retaining the capture lug to engage the cable segments; and the collet may include a quick disconnect mechanism having a base in which the collet is positioned and a spring that biases the base to a closed position of the collet.

In accordance with still another embodiment of the invention there is provided a method of controlling a medical instrument that has a proximal end including a control handle and a distal end including a distal tool, with the control handle and distal tool being intercoupled by an elongated instrument shaft and the tool actuated from a tool control cable. The method includes providing proximal and distal movable members that respectively intercouple the proximal control handle and the distal tool with the instrument shaft, the proximal and distal movable members being intercoupled so that a motion at the proximal movable member controls the distal movable member, dividing the tool control cable into separate cable segments and interlocking the separate cable segments so that the tool control cable is operable. A further step may include manually controlling, from the proximal end of the instrument, the rotation of the distal tool. about its longitudinal distal tool axis.

In accordance with another embodiment of the invention there is provided an instrument having a proximal control handle and a distal tool that are intercoupled by an elongated instrument shaft, proximal and distal movable members that respectively intercouple the proximal control handle and the distal tool with the instrument shaft, means disposed between the movable members so that a motion at the proximal movable member controls the distal movable member and, in turn, the distal tool, means supported at the handle for controlling the distal tool including a tool control cable that extends between the handle and distal tool, the tool control cable including separate control cable segments that are adapted to have one of an engaged state and a dis-engaged state.

In accordance with still other aspects of the present invention there is provided a control member at the control handle and manipulable by a user to control, via the proximal and distal movable members, the rotation of the distal tool about its distal tool axis; the proximal motion member can be disconnected from the control handle when the control cable segments are in their dis-engaged state; including a coupler for selectively engaging or disengaging the separable cable segments; wherein the coupler may include a collet attached to one of the cable segments and a capture lug on the other of the cable segments, the collet for retaining the capture lug to engage the cable segments; and wherein the collet may include a quick disconnect mechanism having a base in which the collet is positioned and a spring that biases the base to a closed position of the collet.

### BRIEF DESCRIPTION OF THE DRAWINGS

Numerous other advantages can be realized in accordance with the present invention by referring to the accompanying drawings, in which:

FIG. 1 is a perspective view of a surgical instrument constructed in accordance with the present invention with the disposable shaft portion and reusable handle portion engaged;

FIG. 2 is a side elevation view of the instrument of FIG. 1;

FIG. 3 is a top plan view of the instrument of FIG. 1;

FIG. 4 is a side elevation view like the view of FIG. 2 but illustrating the handle in an open position and the with the shaft portion being removed from the handle;

FIG. 5 is an enlarged cross-sectional view of the main handle portion and the proximal shaft portion in a closed and locked position;

FIG. 6 is a cross-sectional view of the instrument illustrated in FIGS. 1-5 as taken along line 6-6 of FIG. 5;

FIG. 7 is a cross-sectional view of the instrument illustrated in FIGS. 1-5 as taken along line 7-7 of FIG. 6;

FIG. 8 is a cross-sectional view of the instrument illustrated in FIGS. 1-5 as taken along line 8-8 of FIG. 6;

FIG. 9 is a fragmentary enlarged cross-sectional view of the instrument with the handle in an open position;

FIG. 10 is a cross-sectional view similar to the view of FIG. 9, but illustrating the shaft portion in the process of being removed;

FIG. 11 is a cross-sectional view of an alternate embodiment in which the proximal cable portion 38B has been replaced by a linkage mechanism; and

FIG. 12 is a fragmentary enlarged cross-sectional view of the instrument of FIG. 11 with the handle in an open position.

### DETAILED DESCRIPTION

The present invention is illustrated in the drawings as a surgical instrument that has two portions such that a detachable instrument shaft portion may be disposable and a re-usable handle portion may be sterilized and reused numerous times. This allows for a higher quality instrument handle portion while keeping the overall price of the instrument reasonable. The instrument may also be considered as reposable, meaning that the shaft portion can be re-used a limited number of times, while the handle portion is re-used a significant number of times.

The instrument of the present invention may be used to perform minimally invasive procedures. “Minimally invasive procedure,” refers herein to a surgical procedure in which a surgeon operates through a small cut or incision, the small incision being used to access the operative site. In one embodiment, the incision length ranges from 1 mm to 20 mm in length, preferably from 5 mm to 10 mm in length. This procedure contrasts those procedures requiring a large cut to access the operative site. Thus, the flexible instrument is preferably used for insertion through such small incisions and/or through a natural body lumen or cavity, so as to locate the instrument at an internal target site for a particular surgical or medical procedure. The introduction of the surgical instrument into the anatomy may also be by percutaneous or surgical access to a lumen, vessel or cavity, or by introduction through a natural orifice in the anatomy.

In addition to use in a laparoscopic procedure, the instrument of the present invention may be used in a variety of other medical or surgical procedures including, but not limited to, colonoscopic, upper GI, arthroscopic, sinus, thoracic, prostate, transvaginal, orthopedic and cardiac procedures. Depending upon the particular procedure, the instrument shaft may be rigid, semi-rigid or flexible.

Although reference is made herein to a “surgical instrument,” it is contemplated that the principles of this invention also apply to other medical instruments, not necessarily for surgery, and including, but not limited to, such other implements as catheters, as well as diagnostic and therapeutic instruments and implements.

There are a number of unique features embodied in the instrument that is described herein. For example, there is provided a locking mechanism that is constructed using a ball and socket arrangement disposed about the proximal motion member that follows the bending action and in which an annular cinch ring is used to retain the ball and socket arrangement in a fixed particular position, and thus also maintain the proximal and distal bendable members in a particular bent condition, or in other words, locked in that position. The cinch ring includes a locking lever that is conveniently located adjacent to the instrument handle and that is easily manipulated to lock and unlock the cinch ring and, in turn, the position of the end effector. The cinch ring is also preferably rotatable to that the locking lever can be positioned conveniently or can be switched (rotated) between left and right hand use. This lock control allows the surgeon one less degree of freedom to concentrate on when performing certain tasks. By locking the bendable sections at a particular position, this enables the surgeon to be more hands-free for controlling other degrees of freedom of the instrument such as manipulation of the rotation knob to, in turn, control the orientation of the end effector.

A main feature of the present invention relates to the ability of the instrument to be partially disposable and partially re-useable. In that way the instrument cost can be substantially reduced as it is not necessary to replace the entire instrument for each medical procedure. In one embodiment of the present invention a disconnect means is provided at the handle where at least the distal motion member, tool, instrument shaft and proximal motion member are separable from the handle of the instrument. This enables the distal components to be engageable and dis-engageable from the handle. The handle portion of the instrument is re-useable and thus the cost of that part of the instrument is essentially spread over several instrument uses.

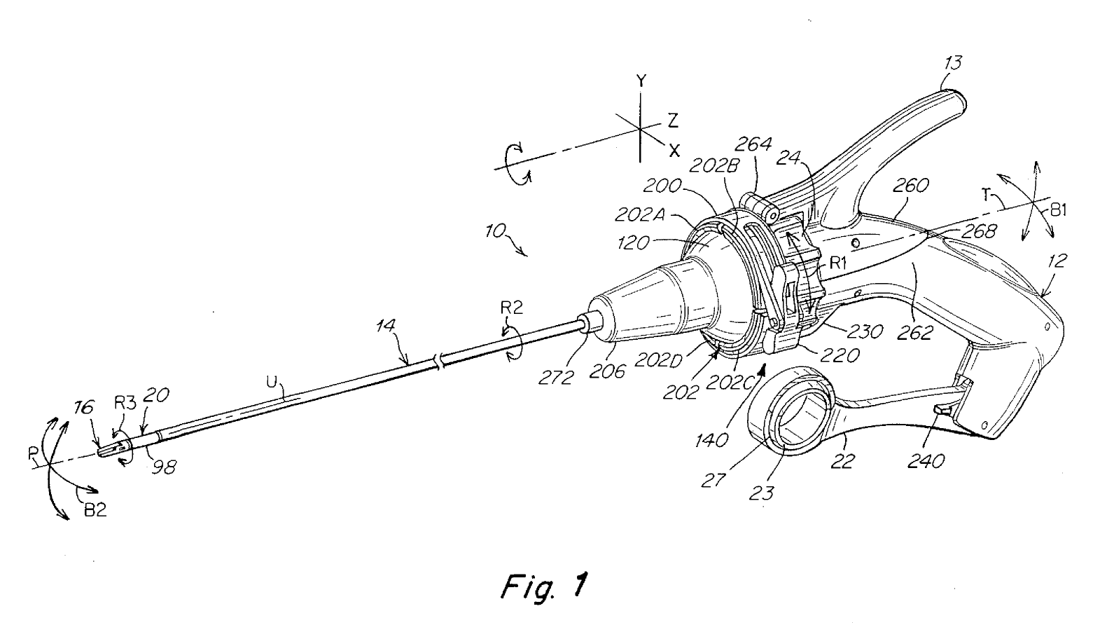

FIG. 1 is a perspective view of one embodiment of the surgical instrument 10 of the present invention. In this surgical instrument both the tool and handle motion members or bendable members are capable of bending in any direction. They are interconnected via cables (preferably four cables) in such a way that a bending action at the proximal member provides a related bending at the distal member. The proximal bending is controlled by a motion or deflection of the control handle by a user of the instrument. In other words the surgeon grasps the handle and once the instrument is in position any motion (deflection) at the handle immediately controls the proximal bendable member which, in turn, via cabling controls a corresponding bending or deflection at the distal bendable member. This action, in turn, controls the positioning of the distal tool.

The proximal member is preferably generally larger than the distal member so as to provide enhanced ergonomic control. In the illustrated embodiment the ratio of proximal to distal bendable member diameters may be on the order of three to one. In one version in accordance with the invention there may be provided a bending action in which the distal bendable member bends in the same direction as the proximal bendable member. In an alternate embodiment the bendable, tamable or flexible members may be arranged to bend in opposite directions by rotating the actuation cables through 180 degrees, or could be controlled to bend in virtually any other direction depending upon the relationship between the distal and proximal support points for the cables.

As has been noted, the amount of bending motion produced at the distal bending member is determined by the dimension of the proximal bendable member in comparison to that of the distal bendable member. In the embodiment described the proximal bendable member is generally larger than the distal bendable member, and as a result, the magnitude of the motion produced at the distal bendable member is greater than the magnitude of the motion at the proximal bendable member. The proximal bendable member can be bent in any direction (about 360 degrees) controlling the distal bendable member to bend in either the same or an opposite direction, but in the same plane at the same time. Also, as depicted in FIG. 1, the surgeon is able to bend and roll the instrument's tool about its longitudinal axis to any orientation simply by rolling the axial rotation knob 24 about a rotation direction indicated in FIG. 1 by the rotation arrow R1. In addition the entire instrument itself can be rotated by manual rotation of the handle.

In this description reference is made to bendable members. These members may also be referred to as turnable members, bendable sections or flexible members. In the descriptions set out herein, terms such as “bendable section,” “bendable segment,” “bendable member,” or “turnable member” refer to an element of the instrument that is controllably bendable in comparison to an element that is pivoted at a joint. The term “movable member” is considered as generic to bendable sections and joints. The bendable elements of the present invention enable the fabrication of an instrument that can bend in any direction without any singularity and that is further characterized by a ready capability to bend in any direction, all preferably with a single unitary or uni-body structure. A definition of a “unitary” or “uni-body” structure is—a structure that is constructed only of a single integral member and not one that is formed of multiple assembled or mated components.

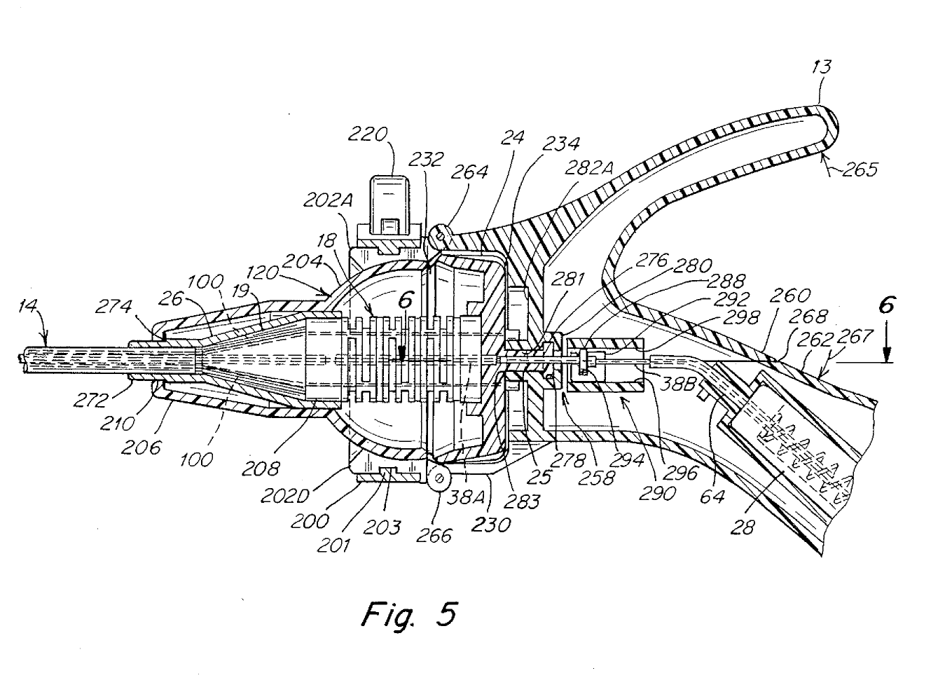

A definition of these bendable members is—an instrument element, formed either as a controlling means or a controlled means, and that is capable of being constrained by tension or compression forces to deviate from a straight line to a curved configuration without any sharp breaks or angularity. Bendable members may be in the form of unitary structures, such as of the type shown herein in FIG. 5 for the proximal bendable member, may be constructed of engageable discs, or the like, may include bellows arrangements or may comprise a movable ring assembly. For several forms of bendable members refer to co-pending applications Ser. No. 11/185,911 filed on Jul. 20, 2005; 11/505,003 filed on Aug. 16, 2006 and 11/523,103 filed on Sep. 19, 2006, all of which are hereby incorporated by reference herein in their entirety.

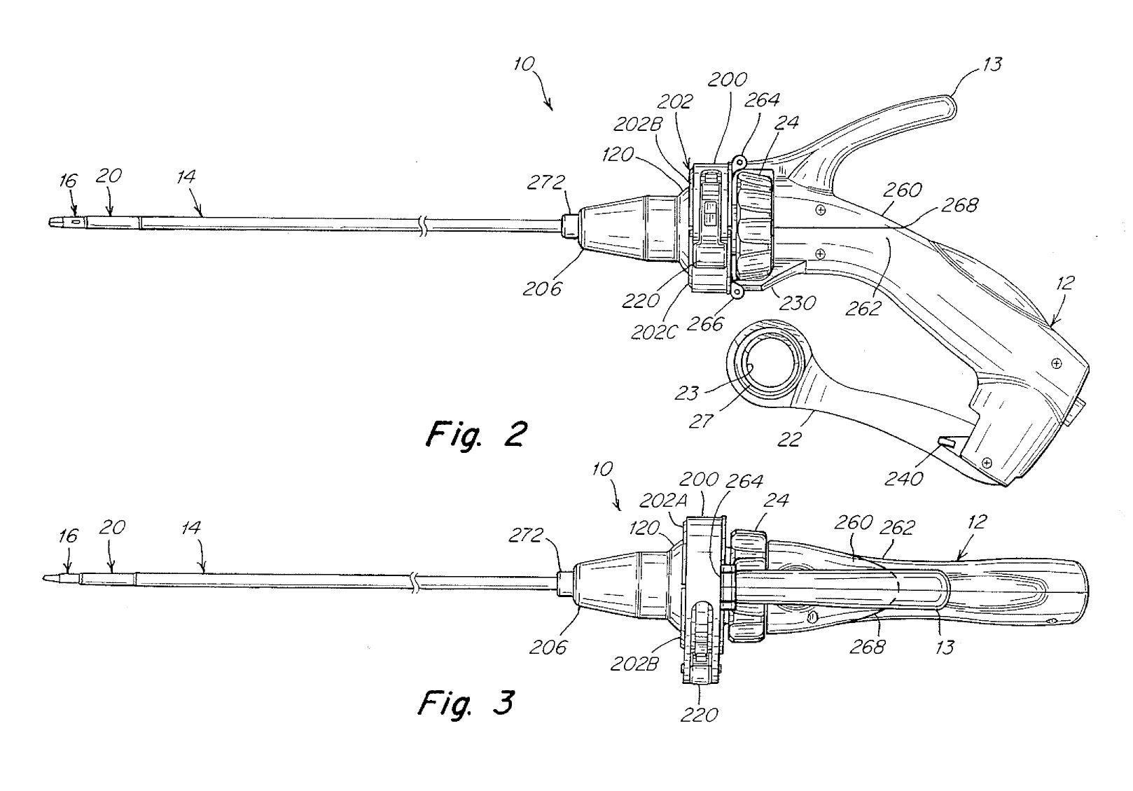

FIG. 1 shows a preferred embodiment of the instrument of the present invention. Further details are illustrated in FIGS. 2 through 10. FIG. 1 depicts the surgical instrument 10 in a perspective view, and as may be positioned during a surgical procedure. For example, the instrument may be used for laparoscopic surgery through the abdominal wall. For this purpose there is provided an insertion site at which there is disposed a cannula or trocar. The shaft portion 14 of the instrument 10 is adapted to pass through the cannula or trocar so as to dispose the distal end of the instrument at the operative site. The end effector 16 is depicted in FIG. 1. The embodiment of the instrument shown in FIG. 1 is typically used with a sheath 98 covering the distal member 20 to keep bodily fluids from entering the distal bendable member 20.

A rolling motion can be carried out with the instrument of the present invention. This can occur, as indicated previously, by virtue of the rotation of the rotation knob 24 relative to the handle 12 about a longitudinal shaft axis. This is represented in FIG. 1 by the rotation arrow R1. When the rotation knob 24 is rotated, in either direction, this causes a corresponding rotation of the instrument shaft 14. This is depicted in FIG. 1 by the rotational arrow R2. This same motion also causes a rotation of the distal bendable member and end effector 16, about an axis that corresponds to the instrument tip, depicted in FIG. 1 as about the longitudinal tip or tool axis P. In FIG. 1 refer to the rotational arrow R3 at the tip of the instrument, The instrument can also be rotated by the surgeon by rotating the handle in either direction.

Any rotation of the rotation knob 24 while the instrument is locked maintains the instrument tip at the same angular position, but rotates the orientation of the tip (tool). For a further explanation of the tip rotational feature refer to co-pending application Ser. No. 11/302,654, filed on Dec. 14, 2005, particularly FIGS. 25-28, which is hereby incorporated by reference herein in its entirety. Thus tip rotation is about a longitudinal tip axis P. Also, this rotation at the tip of the instrument occurs upon rotation of the knob 24 and regardless of the orientation (angle) of end effector as controlled by the interaction of the bendable members from the handle.

The handle 12, via proximal bendable member 18, may be tilted at an angle to the instrument shaft longitudinal center axis. This tilting, deflecting or bending may be considered as in the plane of the paper. By means of the cabling this action causes a corresponding bend at the distal bendable member 20 to a position wherein the tip is directed along an axis and at a corresponding angle to the instrument shaft longitudinal center axis. The bending at the proximal bendable member 18 is controlled by the surgeon from the handle 12 by manipulating the handle in essentially any direction including in and out of the plane of the paper in FIG. 1. This manipulation directly controls the bending at the proximal bendable member. For further descriptions relating to the bending refer to co-pending application Ser. Nos. 11/528,134 filed on Sep. 27, 2006 and 11/649,352 filed on Jan. 2, 2007, both of which are hereby incorporated by reference in their entirety.

Thus, the control at the handle is used to bend the instrument at the proximal motion member to, in turn, control the positioning of the distal motion member and tool. The “position” of the tool is determined primarily by this bending or motion action and may be considered as the coordinate location at the distal end of the distal motion member. Actually, one may consider a coordinate axis at both the proximal and distal motion members as well as at the instrument tip. This positioning is in three dimensions. Of course, the instrument positioning is also controlled to a certain degree by the ability of the surgeon to pivot or rotate the instrument at the incision point or at the cannula or trocar. The “orientation” of the tool, on the other hand, relates to the rotational positioning of the tool, from the proximal rotation control member, about the illustrated distal tip or tool axis P.

In the drawings a set of jaws is depicted, however, other tools or devices may be readily adapted for use with the instrument of the present invention. These include, but are not limited to, cameras, detectors, optics, scope, fluid delivery devices, syringes, etc. The tool may include a variety of articulated tools such as: jaws, scissors, graspers, needle holders, micro dissectors, staple appliers, tackers, suction irrigation tools and clip appliers. In addition, the tool may include a non-articulated tool such as: a cutting blade, probe, irrigator, catheter or suction orifice.

The surgical instrument of FIG. 1 shows a preferred embodiment of a surgical instrument 10 according to the invention in use and may be inserted through a cannula at an insertion site through a patient's skin. Many of the components shown herein, such as parts of the instrument shaft 14, end effector 16, distal bendable member 20, and proximal bendable member 18 may be similar to and interact in the same manner as the instrument components described in the co-pending U.S. application Ser. No. 11/185,911 filed on Jul. 20, 2005 and hereby incorporated by reference herein in its entirety. Many other components shown herein, particularly at the handle end of the instrument may be similar to components described in the co-pending U.S. application Ser. No. 11/528,134 filed on Sep. 27, 2006 and hereby incorporated by reference herein in its entirety. Also incorporated by reference in their entirety are U.S. application Ser. No. 10/822,081 filed on Apr. 12, 2004; U.S. application Ser. No. 11/242,642 filed on Oct. 3, 2005 and U.S. application Ser. No. 11/302,654 filed on Dec. 14, 2005, all commonly owned by the present assignee.

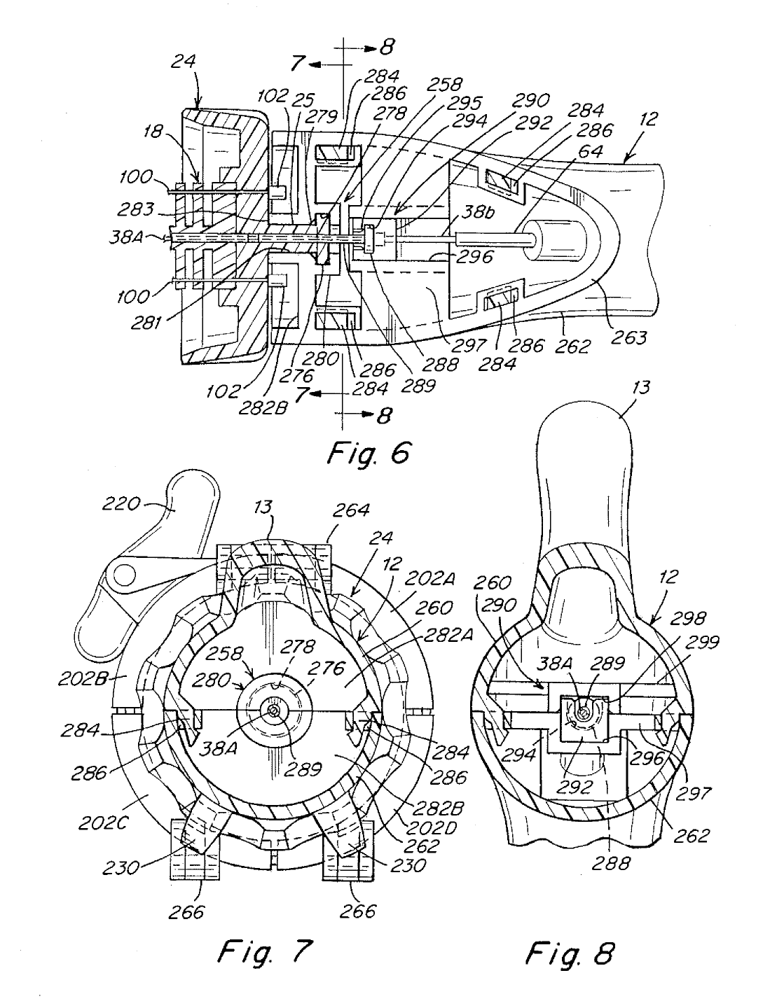

The control between the proximal bendable member 18 and distal bendable member 20 is provided by means of the bend control cables 100. FIGS. 5 and 6 illustrate the cables 100. In the illustrated embodiment four such control cables 100 may be provided in order to provide the desired all direction bending. When using four cables they would be separated by 90 degrees. However, in other embodiments of the present invention fewer or less numbers of bend control cables may be used. The bend control cables 100 extend through the instrument shaft 14 and through the proximal and distal bendable members. The bend control cables 100 may be constrained along substantially their entire length so as to facilitate both “pushing” and “pulling” action as discussed in further detail in the aforementioned co-pending application Ser. No. 11/649,352 filed on Jan. 2, 2007. The cables 100 may also be constrained as they pass over the conical cable guide portion 19 of the proximal bendable member 18, and through the proximal bendable member itself Refer to FIGS. 5 and 6 for more details of the cable termination at the proximal end thereof, including the termination lugs or wire crimps illustrated at 102 in FIG. 6.

The locking means interacts with the ball and socket arrangement to lock and unlock the positioning of the cables which, in turn, control the angle of the proximal bendable member and thus the angle of the distal bendable member and end effector. This lock control allows the surgeon one less degree of freedom to concentrate on when performing certain tasks. By locking the bendable sections at a particular position, this enables the surgeon to be more hands-free for controlling other degrees of freedom of the instrument such as manipulation of the rotation knob 24 and, in turn, orientation of the end effector. Although the locking mechanism freezes the position of the bend, it still enables rotation (orientation) of the instrument tip at the tool 16 about axis P.

The instrument shown in FIG. 1 is considered as of a pistol grip type. However, the principles of the present invention may also apply to other forms of handles such as a straight in-line handle. In FIG. 1 there is shown a jaw clamping or actuation mechanism that is comprised mainly of the lever 22 which may have a single finger hole 23 in the gimbaled ball 27. The ball 27 is mounted at the free end of the lever 22. The surgeon uses the ball 27 for controlling the lever 22. There may also be provided a related release function controlled either directly by the lever 22 or a separate release button. The release function is used to release the actuated or closed tool or end effector.

In the instrument that is illustrated the handle end of the instrument may be tipped or deflected in any direction as the proximal bendable member is constructed and arranged to preferably enable full 360 degree bending. This movement of the handle relative to the instrument shaft bends the instrument at the proximal bendable member 18. This action, in turn, via the bend control cables 100, bends the distal bendable member in the same direction. As mentioned before, opposite direction bending can be used by rotating or twisting the control cables through 180 degrees from one end to the other end thereof.

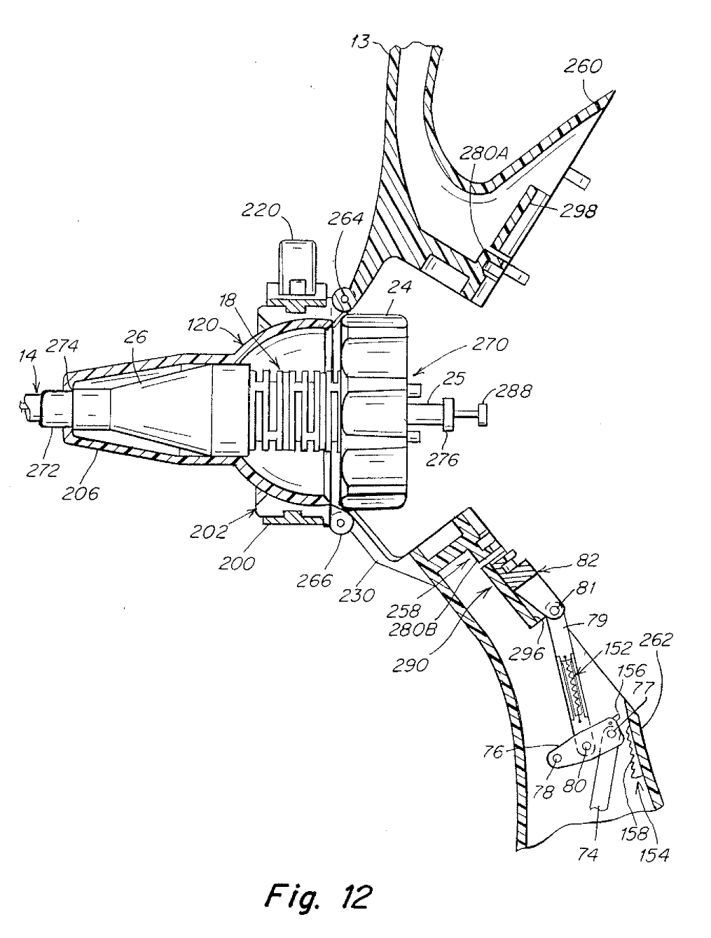

In the main embodiment described herein, the handle 12 is in the form of a pistol grip and includes a horn 13 to facilitate a comfortable interface between the action of the surgeon's hand and the instrument. In this embodiment the horn has the other function of providing part of one of the members that define a clamshell closure (260, 262) for retaining the tool control cable, as described in more detail later. The tool actuation lever 22 is shown in FIG. 1 pivotally attached at the base of the handle. The lever 22 actuates a slider 28 (see FIG. 5) that controls the tool actuation cable 38 that extends from the slider to the distal end of the instrument. The cable 38 controls the opening and closing of the jaws, and different positions of the lever control the force applied at the jaws. The cable is depicted, for example, in FIGS. 5 and 6 as including distal cable portion 38A and proximal cable portion 38B. In place of the proximal cable portion there may be provided a different type of actuation member such as a linkage arrangement that is able to either pull or release the distal cable portion. One embodiment thereof is shown herein in FIGS. 11 and 12, which are discussed in more detail hereinafter.

The instrument 10 has a handle portion 12 and a detachable shaft portion 14, as shown in FIG. 1. The main components of the instrument may be like that shown in Ser. No. 11/649,352 tiled on Jan. 2, 2007, particularly as to the construction of the bendable members, instrument shaft and end effector. This includes means for enabling rotation of the shaft and proximal bendable member within bearings or bearing surfaces 208 and 210 (FIG. 5). The bearing surface 208 interfaces between the adaptor 26 and the ball 120, while the bearing surface 210 interfaces between the neck portion 206 of the ball member and the distal end of the adaptor 26. The separate portions 12 and 14, or alternatively the assembled instrument, may be sealed in a sterile package or packages prior to storage or shipping.

In the embodiment of the instrument illustrated in FIG. 1 the instrument has an easily replaceable shaft portion that may be disposable after a single surgical procedure. On the other hand the handle portion, which can be more expensive in construction, is able to be re-sterilized such as by being autoclaved for reuse with a new shaft portion having been installed. The replaceable shaft portions may be identical or may mount different end tips so that different end effectors can be used with the same handle portion. The instrument itself may be like that shown in Ser. No. 11/649,352 filed on Jan. 2, 2007, particularly as to the construction of the bendable members, instrument shaft and end effector. In the following description like reference numbers are used.

The following is a description of the many features of the instrument that is illustrated. herein in the drawings. Thus, a bending at the handle in relation to the instrument shaft causes a corresponding bending of the distal bending member 20 and end tool. An angle locking means 140 allows the axis T of the handle 12 to be adjustably bent and locked at an angle B1 to the axis U of shaft 14 resulting in a bend at angle B2 between the axis P of the end effector and the axis U of the instrument shaft 14. Release/lock lever 220 can be flipped to a position where cinch ring 200 is relaxed and the split hub portions 202A-202D are released from their grip of the spherical surface 204 of the ball 120. The ball is then free to oscillate in the split hub 202 in essentially three dimensions by relative motion between the shaft and handle. When the handle axis T is bent at angle B1 in relationship to the axis U of the instrument shaft, cables 100 connecting the respective proximal and distal bendable members 18 and 20 are pulled/pushed a preset ratio resulting in a bend at angle B2 at the distal bendable member 20 which bends axis P of the end effector 16 at an angle to the axis U of shaft 14. The selected angle is then locked in by flipping the release/lock lever 220 and thus tightening up the cinch ring. Turning rotation knob 24, in either the locked or unlocked position, rotates the instrument shaft 14, in the direction of arrow R2 about instrument shaft axis U. That rotation is translated into rotation of the distal bendable member 20 so as to, in turn, rotate the end effector 16 about its axis P, and whether axis P is lined up or at an angle to axis U. This action is maintained about the axis P and not by an orbiting about axis U.

Earlier instruments, such as shown in Ser. Nos. 11/900,417 (Pub. No. 2009/0069842) and 12/006,278 (Pub. No. 2009/0171147) illustrate instruments in which the shaft portion is detachable by means of a front loading arrangement. These two applications are now incorporated by reference herein in their entirety. Although this is an effective means for providing a disposable shaft portion, the arrangement of the present invention as illustrated in the drawings herein represents an improvement to those instruments. The present invention uses a rear loading shaft portion in which the instrument construction is more simplified and thus can be manufactured at a lower cost, for both the handle and shaft portions. The instrument of the present invention uses a set of handle housing sections that can pivot between open and closed positions. In the open position thereof the shaft portion is readily removable from or insertable into the handle. The simple closing of the housing sections locks the shaft portion in place and enables operation of the tool actuation cable from the actuation lever.

Another advantage to the instrument of the present invention is the ability to easily clean the instrument, particularly the handle portion of the instrument. The opening at the rear of the handle portion provides an open structure that is readily accessible and thus easy to clean. Also with the rear loading arrangement of the present invention the shaft portion can in particular be more easily loaded into the handle portion. The shaft portion is readily guided into the handle portion as it is the small distal part of the shaft portion that first engages the handle portion. This tip of the shaft portion makes it quite easy for the surgeon to engage the shaft portion with the handle portion.

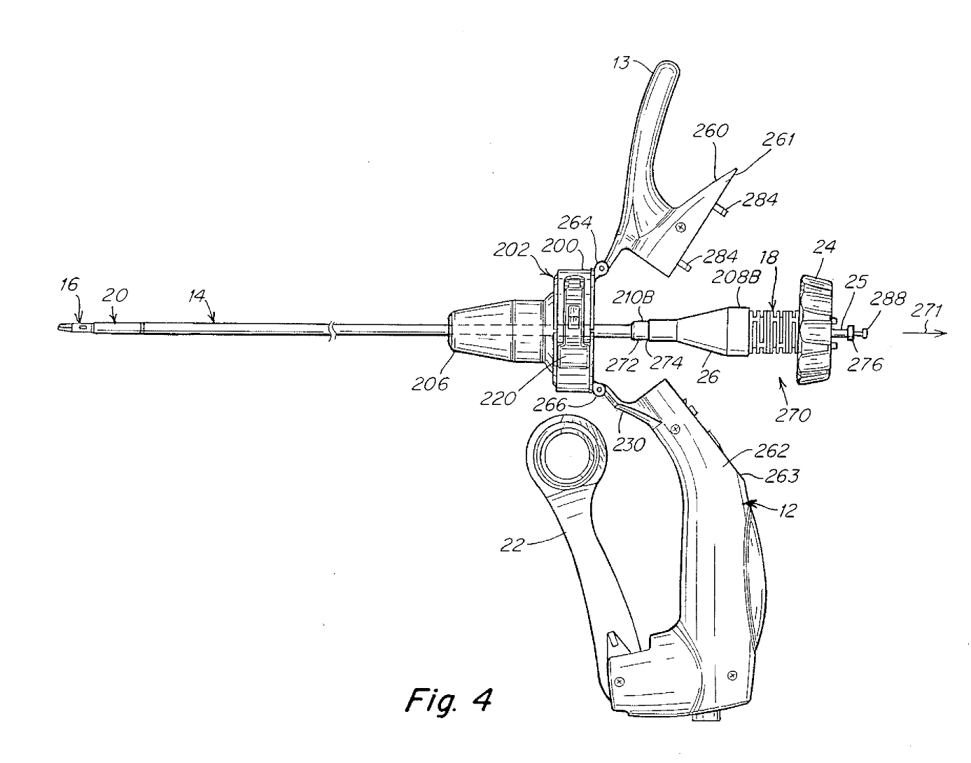

The handle 12 of the instrument 10 is configured to open from the proximal end like a clamshell with a top clamshell portion 260 and a bottom clamshell portion 262. The portions 260, 262 have rims 261 and 263 respectively that mate with each other along the part line 268. Hinges 264 and 266, connected to the split hub 202, allow the clamshell halves to pivot open enough to allow the rotation knob 24 to be easily grasped and the shaft assembly 270 (FIG. 4) to be removed from the handle portion 12. The used shaft assembly which is relatively cheap to make with inexpensive materials can be discarded since it will not lend itself to autoclaving due to bodily fluids that may infiltrate passages in the end effector 16 and distal bendable member 20 even though a protective sheath 98 is used over the distal bendable member. The handle portion 12, which remains clear of the body cavity, can be re-sterilized such as by being autoclaved for many reuses. This makes for an instrument that is cost effective in enabling the use of better materials and engineering to produce a quality instrument.

In accordance with a preferred embodiment of the instrument of the present invention, the handle is easily adaptable to be used in the left or right hands by means of features such as cam lever 240, gimbaled ball 27, finger recess 23 and adjustable cinch ring 200 that can be rotated to positions suitable for left or right thumb manipulation of release/lock lever 220. The cam lever 240 shifts the jaw clamping lever 22 to the left or right of the centerline of the handle to accommodate a left or right handed grip. The gimbaled ball 27 allows the finger recess 23 to line up comfortably on the surgeon's finger. The cinch ring can be rotatably adjusted for use with left or right hand thumbs. The cinch ring 200 has a spline 201 that rides in a circumferential groove 203 in the split hub portions 202A-202D. When the cinch ring 200 is in a relaxed state it can be easily rotated about the split hub to a position comfortable for left or right hand use and is maintained loose enough for the ball to oscillate and/or rotate within split hub 202 without coming loose. When the shaft assembly is to be removed, it is preferred that the cinch ring be locked with the shaft axis U aligned with handle axis T. This stabilizes the split hub 202 on the ball 120 and also lines up the axes P and U for easy removal/insertion of shaft assembly 270 in a straight line along axis Z.

Hinges 264, 266 are anchored to split hub 202 and guide the alignment of the clamshell halves 260, 262 when opening and closing them to lock the sections 202A-202D in place. Expanding hinges (not shown) may also be employed in place of hinges 264, 266 to keep the sections 202A-202D in alignment. Refer to FIG. 7 for an illustration of the hinges 264, 266. FIG. 7 depicts a single hinge 264 bridging between the split hub portions 202A and 202B. FIG. 7 also depicts a pair of hinges 266 one associated with each of the split hub portions 202C and 202D. Other hinge arrangements may also be used, or other means for pivotally supporting the clamshell halves 260, 262

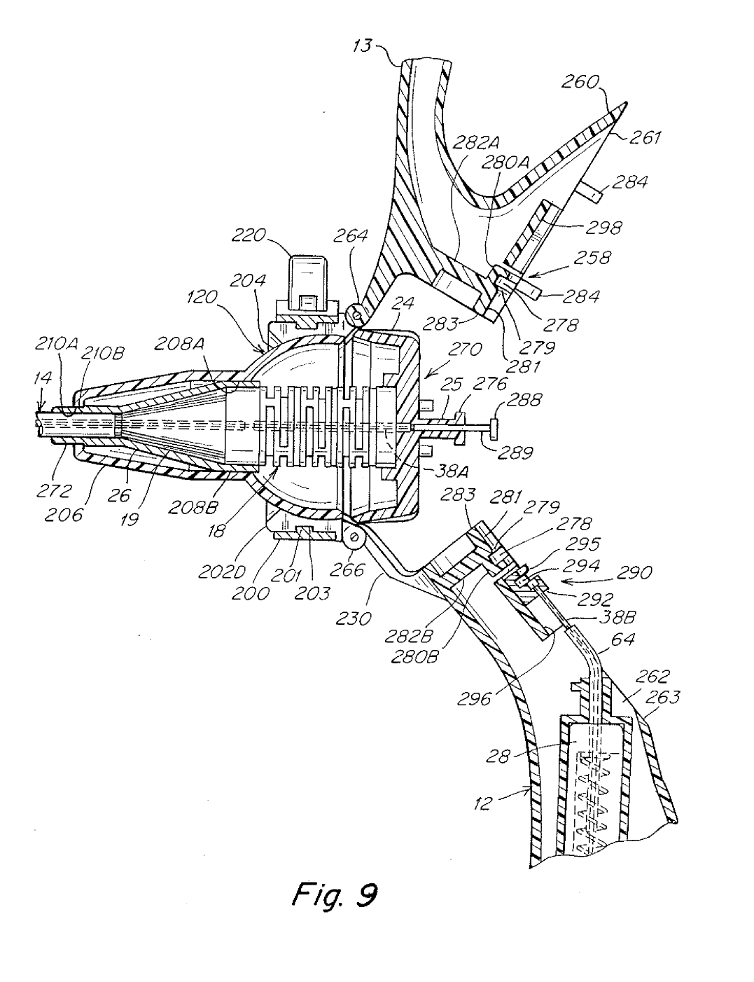

The clamshell is opened by pushing up on the horn 13 and upper clamshell portion 260 in the direction of arrow 265 while applying equal force on the lower clamshell portion 262 of the handle in the direction of arrow 267 as illustrated in FIG. 5. When enough pressure is applied, the male mating features 284 snap out of the female mating features 286 and the upper clam shell portion 260 pivots about hinge 264. The hinge 264 connects split hub segments or sections 202A-202B to the distal end of the horn and upper clamshell portion 260. The lower clamshell portion 262 pivots by means of the hinges 266 which connects split hub segments 202C-202D to the struts 230 of the lower clamshell portion 262. As the clamshell starts to open it releases cable capture/release mechanism 290 and the shaft capture/release mechanism 258. The clamshell portions 260, 262 are swung open to the position illustrated in FIGS. 4 and 9 which exposes the rotational knob 24 to be easily grasped and the shaft assembly 270 removed in the direction of arrow 271.

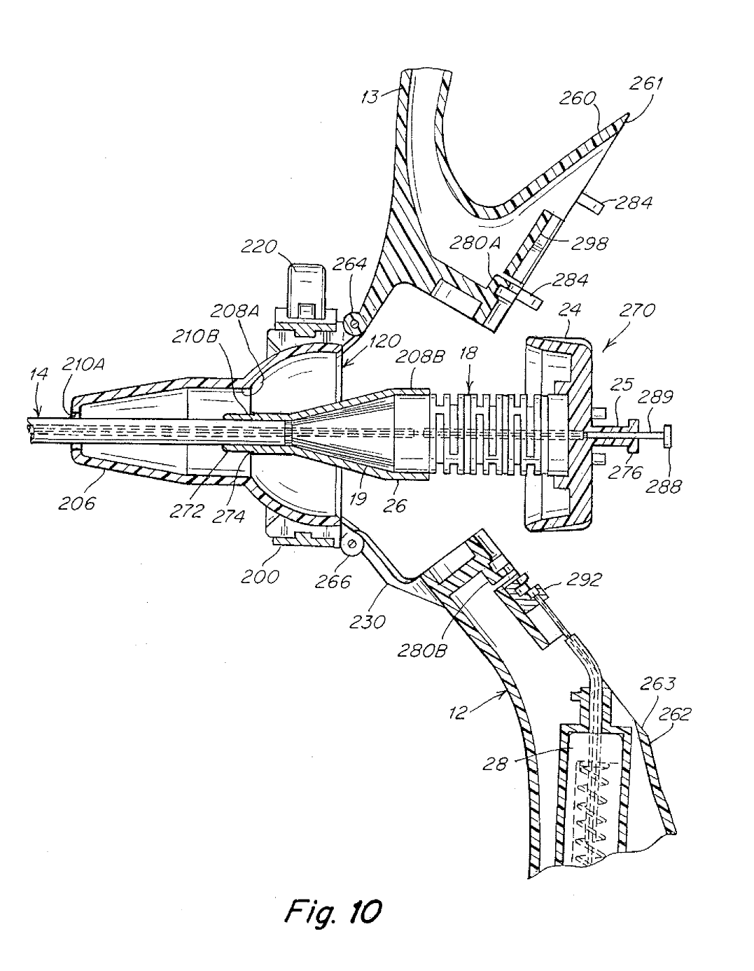

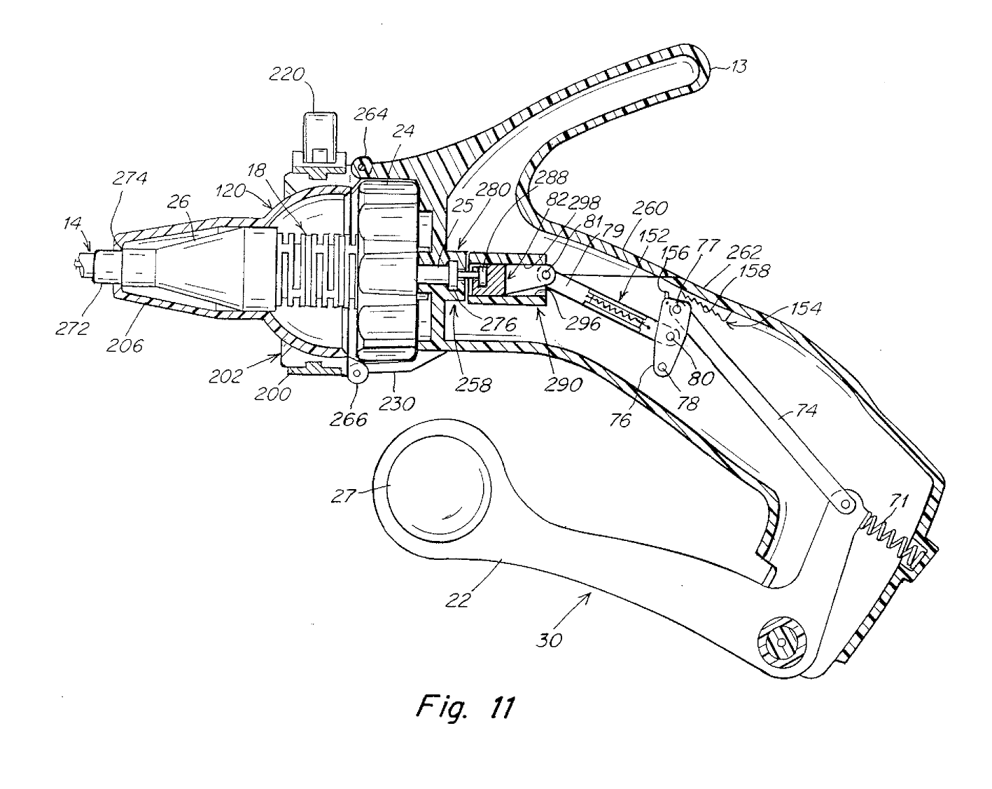

The mechanism 258, when closed, grasps the shoulder 276 which in essence locks in the shaft portion 14. The shoulder or flange 276 is captured in annular groove 278 by the hub 280 (FIG. 5), and in particular by the top and bottom hub halves 280A, 280B (FIG. 9). The hub halves capture the shoulder 276 when the handle portions are closed. The mechanism 290, on the other hand, captures the cable end lug 288 which is part of the actuation cable 38. The capture block 292 allows translation of the actuation cable and is transitional in the guide channels 296, 298 (FIG. 5). The shaft assembly 270, as illustrated in FIGS. 4 and 10, includes at the proximal end thereof, the adaptor 26, proximal bendable member 18 and rotation knob 24. Proximal to the proximal bendable member 18 are illustrated the shoulder 276 on hub 25 and cable end lug 288. The shaft assembly 270 includes at the distal end thereof the shaft 14, distal bendable member 20 and end effector 16.

The replacement shaft assembly 270 can then be inserted into the handle and the clamshell halves 260, 262 closed, capturing the shoulder 276 by the shaft mechanism 258 and capturing the cable end lug 288 by means of the cable mechanism 290. The mating features 284, 286 engage in a snap fit arrangement as depicted in FIG. 7, when the housing portions are closed. These mating features are illustrated as male and female members. However, other forms of mating members may also be used that enable the housing portions to be readily engaged or disengaged. As illustrated in, for example, FIGS. 5 and 9, the portion of the shaft assembly 270 distal of the proximal bending member 18 is free to rotate on bearing surfaces 208A, 210A formed on in inner surface of the neck portion 206 of ball member 120. These bearing surfaces 208A and 210A mate with bearing surfaces 208B, 210B formed on the reduced diameter portion 272 at the distal end of the adapter 26.

The rotational knob 24 is proximal of the proximal bending member 18 and is free to rotate on bearing surfaces 281 on the respective hub portions 280A and 280B (see FIGS. 5 and 9). A shoulder 274 on the reduced diameter portion 272 bears against the end wall of the neck 206 and the distal end 283 of hub 280 bears against the rotational knob 24. Additionally, a shoulder 276 formed on the hub 25 of the rotational knob is captured in the annular groove 278 formed in the hub 280 and prevents linear movement of the shaft so that there is a gap 232 and 234 between the rotational knob and split hub and the rotational knob and handle housing respectively as illustrated in FIG. 5. The gaps 232 and. 234 assure that the rotational knob 24 does not rub against the ball member or handle housing when it is rotated. The hub portions 280A and 280B are formed on radial wall portions 282A and 282B of the clamshell halves 260 and 262 (see FIG. 9).

The push/pull cable 38 that opens and closes the end effector jaws is comprised of two separate sections 38A and 38B. The actuation of the cable 38 is controlled from the actuation lever 22. Cable section 38A extends the length of the shaft assembly from the end effector and terminating proximally in a cable end lug 288 at the end of a tubular portion 289. The tubular portion 289 is slidable within the hub 25 (see the detail of FIG. 6). The tubular portion 289 may be spring loaded (not shown) to assure proper alignment when the lug 288 is captured by block 292 as the clamshell halves are being closed. Such a spring loaded mechanism is described in the aforementioned Ser. No. 11/900,417 (Pub. No. 2009/0069842), and in particular FIG. 29 thereof.

When the clamshell halves are closed, the cable end lug 288 is captured in the cable capture/release mechanism 290 and connected to cable portion 38B by capture block 292 that is affixed to the distal end of cable 38B. The other end of cable 38B which passes through center wire conduit 64 is attached to slider 28 which applies a spring loaded pull to the cable to close the jaws of the end effector when the jaw clamping member or lever 22 is squeezed. The capture block 292 rides in the lower guide channel 296 supported on cross bracket 297 on the lower clamshell portion 262 (FIG. 8). The capture block 292 has a U-shaped channel 294 with a tapered lead in 295 that captures cable end lug 288 when the clamshell halves are closed (see FIGS. 6, 8 and 9). The upper guide channel 298 mounted on cross bracket 299 on the upper clamshell portion 260 abuts the top edge of the lug 288, capturing it in the channel 294. The upper guide channel 298 also abuts the top of the capture block to keep it from riding up in the lower guide channel 296. The cable end lug 288 is free to rotate within U-shaped channel 294 when the rotational knob is rotated.

In the embodiment that is illustrated herein, as mentioned previously, the removable shaft portion is engaged with the handle portion of the instrument from the rear of the instrument, as illustrated in, for example, FIG. 10. This rear loading enables the instrument to be manufactured at less expense and less complexity. It is preferred that the shaft portion include the rotation knob as the rotation knob then functions as an easy place for the user to grasp and withdraw the shaft portion, or for easy insertion of the shaft portion. Thus, the shaft portion preferably includes, as a unitary piece, the distal end effector, the instrument shaft, the proximal bendable member and the rotation knob. What also lends itself to a simplified instrument is the preferred handle construction which includes separate hinged handle portions, as illustrated herein. In another version of the instrument of the present invention the ball member may be removable as a part of the shaft portion, and thus integral with the shaft portion.

In another version of the present invention the proximal cable portion 38B may be replaced by a different mechanism. that is adapted to pull the distal cable portion 38A. FIGS. 11 and 12 schematically depict this in the form of a linkage mechanism that essentially replaces the proximal cable portion 38B depicted in earlier embodiments described herein. The actuation is still controlled from the jaw clamping or actuation means 30, which via the linkage mechanism controls the carriage 82, which, in turn, retains and controls a proximal end of the end effector actuation cable. The carriage 82 may capture the cable end lug 288, as depicted in FIG. 11. A linkage mechanism of the type shown in FIG. 11 herein is similar to the linkage mechanism disclosed in co-pending application Ser. No. 12/006,278 filed on Dec. 31, 2007 and hereby incorporated by reference in its entirety.

The jaw clamping or actuation means 30 is comprised mainly of the lever 22 which may have a single finger hole in a gimbaled ball 27. The ball 27 is mounted at the free end of the lever 22. The surgeon uses the ball 27 for controlling the lever 22. In an alternate embodiment, the ball 27 is optional and in its place is a simple through or blind hole at the free end of the lever 22. There may also be provided a related release function controlled either directly by the lever 22 or a separate release button. When the lever 22 is squeezed the carriage 82 is pulled proximally. This action initiates the pulling of the cable. The further squeezing of the lever 22 toward the handle results in the operation of the ratcheting means 154. The lever 22 can then be fully squeezed to release the ratcheting member 154 and the cable engagement means. This action returns the carriage 82 under bias from the spring 71.

A compensation means 152 may be used to provide a bias force while at the same time accommodating different size needles or other objects at the end effector. The compensation means or member is comprised primarily of a link 79 that is constructed. of two relative sliding portions. The link 79 may be supported in a guide allowing the link 79 to be biased proximally by means of an internal spring. One end of the link 79 is supported from crank 76 at pin 80 while the opposite end is supported from carriage 82 at pin 81. Crank 76 pivots at pin 78. Link 74 is attached to crank 76 at pin 77, and pin 80 supports link 79 from crank 76. When the lever 22 is squeezed the jaws of the end effector may close on an object.

The ratchet mechanism 154 is comprised of a spring loaded pawl 156 acting in a one way ratcheting action on rack 158. Again, refer to Ser. No. 12/006,278 for further details of the operation of the ratchet mechanism. The rack 158 is secured to an inner surface of the handle. in FIG. 11 it is noted that the pawl 156 is not yet engaged with the rack 158. The pawl is adapted to move along the rack until it clears the rack. The pawl 156 is then free to pivot past the teeth of the rack 158 and thus release the crank 76 to be returned to the start position by lever return spring 71. Once the pawl passes the end of its travel it automatically returns wider control of the return spring 71.

The embodiment shown in FIGS. 11 and 12 is also one in which the housing is provided in separate top and bottom clamshell portions 260, 262 that can be separated in order to permit the shaft portion to be inserted or withdrawn. Thus, the handle is a break-away handle in which the separate portions can be opened or closed, and wherein, in the open position, the shaft portion can be inserted or withdrawn. This is a far more simple arrangement than one in which the shaft portion is front mounted. The overall instrument structure, for a front load shaft portion, has to be more complex in order to properly receive the shaft portion and guide the shaft portion into the handle portion. On the other hand, the instrument construction can be more simplified in an instrument in which the shaft portion is rear loaded. This means that for a rear loaded instrument the instrument can be made more inexpensively.

In FIGS. 11 and 12 one version of a linkage mechanism is illustrated. However, in other embodiments many other linkage mechanisms can be substituted. The common thread between these mechanisms is that some type of an actuation lever, or the like, is used and coupled by some mechanism to control the distal actuation cable that, in turn, controls the operation of the end effector. Another alternate embodiment is one in which, rather than having opposed hinges to open the handle portion, one can provide a single hinge on one side only and provide a cut-away section on the other side so that the shaft portion can be inserted into or withdrawn from the handle portion.

Having now described a limited number of embodiments of the present invention it should now be apparent to one skilled in the art that numerous other embodiments and modifications thereof are contemplated as falling within the scope of the present invention as defined by the appended claims.

What is claimed is:

1. A surgical instrument comprising:
an instrument shaft having proximal and distal ends;
a tool disposed from the distal end of the instrument shaft;
a control handle coupled from. the proximal end of the instrument shaft;
a distal motion member for coupling the distal end of said instrument shaft to said tool;
a proximal motion member for coupling the proximal end of said instrument shaft to said handle;
actuation means extending between said distal and proximal motion members for coupling motion of said proximal motion member to said distal motion member for controlling the positioning of said tool;
and an actuation cable extending from said handle to said tool for controlling the actuation of the tool;
said actuation cable separated into two inter-engagable cable segments that enable the proximal motion member to be disconnected from the control handle;
said control handle including separate housing sections that are each pivotally supported from a main housing section between open and closed positions.

2. The surgical instrument of claim 1 further including a rotation means disposed adjacent the control handle and rotatable relative to the control handle for causing a corresponding rotation of the instrument shaft and tool.

3. The surgical instrument of claim 2 wherein at least said proximal motion member comprises a proximal bendable member, said rotation means comprises a rotation knob that is adapted to rotate the tool about a distal tool roll axis and said rotation knob is disposed between said control handle and proximal bendable member.

4. The surgical instrument of claim 1 wherein at least said instrument shaft and tool comprise a shaft portion and at least said control handle comprises a handle portion, said shaft portion being removably loadable to said handle portion from the rear of the handle portion and through the separated housing sections.

5. The surgical instrument of claim 4 wherein the removable shaft portion also includes a rotation knob that is fixed with the proximal motion member.

6. The surgical instrument of claim 1 including a locking mechanism for fixing the position of the tool at a selected position and having locked and unlocked states, said locking mechanism including a ball and socket arrangement disposed about said proximal motion member and a cinch member for locking said ball and socket arrangement.

7. The surgical instrument of claim 6 wherein the socket member comprises a split hub socket and said cinch member closes said split socket to lock the socket on the ball.

8. The surgical instrument of claim 1 wherein the separate housing sections include a top portion having a first hinge connected from the main housing section and a bottom portion having a second hinge connected from the main housing section.

9. The surgical instrument of claim 8 wherein the hinges are constructed and arranged so that the top and bottom portions hinge in opposite directions, at least said instrument shaft and tool comprising a shaft portion and at least said control handle comprising a handle portion, said shaft portion being removably loadable to said handle portion from the rear of the handle portion and through the separated hinged housing sections.

10. In a medical instrument having a proximal control handle and a distal tool that are intercoupled by an elongated instrument shaft that is meant to pass internally of an anatomic body, proximal and distal movable members that respectively intercouple said proximal control handle and said distal tool with said instrument shaft, cable control means disposed between said movable members, an actuation member at said handle for controlling said distal tool through said movable members, a coupler for selectively engaging or disengaging separable segments of the actuation member, and a handle housing having oppositely disposed housing portions that are pivotable from a main housing portion so as to assume either an open position or a closed position.

11. The medical instrument of claim 10 wherein at least said proximal movable member, instrument shaft and distal tool form a shaft portion of the instrument and at least the control handle forms a handle portion of the instrument, in the open position of the handle housing the shaft portion being engageable and dis-engageable with the handle housing, and in the closed position of the handle housing the shaft portion being locked to the handle portion.

12. The medical instrument of claim 11 including a rotation knob for rotating at least the proximal movable member and instrument shaft, the rotation knob also forming a part of the shaft portion that is engageable and dis-engageable with the handle portion.

13. The medical instrument of claim 12 wherein said coupler includes a shaft release mechanism and a cable release mechanism.

14. The medical instrument of claim 13 wherein said shaft release mechanism includes a shaft assembly for supporting the rotation knob, said cable release mechanism includes a capture mechanism for joining the separable segments and supported for limited translation, and wherein the separate segments are cable segments.

15. A method of controlling a medical instrument that has a proximal end including a control handle and a distal end including a distal tool, said control handle and distal tool being intercoupled by an elongated instrument shaft and said tool actuated from a tool control cable, said method including providing proximal and distal movable members that respectively intercouple said proximal control handle and said distal tool with said instrument shaft, said proximal and distal movable members being intercoupled so that a motion at said proximal movable member controls said distal movable member, dividing the tool control cable into separate cable segments, interlocking the separate cable segments so that the tool control cable is operable, manually controlling, from the proximal end of the instrument, the rotation of said distal tool about its longitudinal distal tool axis, and providing a handle housing having oppositely disposed housing portions that are pivotable from a main housing portion so as to assume either an open position or a closed position.

16. The method of claim 15 wherein at least said proximal movable member, instrument shaft and distal tool form a shaft portion of the instrument and at least the control handle forms a handle portion of the instrument, in the open position of the handle housing the shaft portion being engageable and dis-engageable with the handle housing, and in the closed position of the handle housing the shaft portion being locked to the handle portion.

17. An instrument having a proximal control handle and a distal tool that are intercoupled by an elongated instrument shaft, proximal and distal movable members that respectively intercouple said proximal control handle and said distal tool with said instrument shaft, means disposed between said movable members so that a motion at said proximal movable member controls said distal movable member and, in turn, the distal tool, means supported at the handle for controlling the distal tool including a tool control cable that extends between the handle and distal tool, said tool control cable including separate control cable means that are adapted to have one of an engaged state and a dis-engaged state.

18. The instrument of claim 17 including a control member at said control handle and manipulable by a user to control, via said proximal and distal movable members, the rotation of said distal tool about its distal tool axis.

19. The instrument of claim 17 wherein the proximal motion member can be disconnected from the control handle when the control cable segments are in their dis-engaged state.

20. The instrument of claim 19 including a coupler for selectively engaging or disengaging the separable cable segments.

21. The instrument of claim 19 wherein said coupler includes a collet attached to one of said cable segments and a capture lug on the other of the cable segments, said collet for retaining said capture lug to engage the cable segments.

22. The instrument of claim 21 wherein said collet includes a quick disconnect mechanism having a base in which the collet is positioned and a spring that biases the base to a closed position of the collet.

23. A surgical instrument that is comprised of a handle portion and a removable shaft portion, and in which the handle portion includes a control handle having separable clamshell portions that can assume open and closed positions, said shaft portion including an instrument shaft having proximal and distal ends, a tool disposed from the distal end of the instrument shaft, a distal bendable member for coupling the distal end of said instrument shaft to said tool, a proximal bendable member for coupling the proximal end of said instrument shaft to said handle, actuation. means extending between said distal and proximal motion members for coupling motion of said proximal motion member to said distal motion member for controlling the positioning of said tool, and an actuation cable extending from said handle to said tool for controlling the actuation of the tool, said shaft portion being rear mounted and insertable into said handle portion between the respective clamshell portions of the handle.

24. The instrument of claim 23 wherein said handle includes a linkage mechanism for controlling the actuation cable.

25. The instrument of claim 23 wherein the actuation cable is separated into two inter-engagable cable segments that enable the proximal motion member to be disconnected from the control handle.

26. The instrument of claim 23 wherein at least one of the clamshell portions are pivotally supported from the handle between open and closed positions.

27. The instrument of claim 26 wherein the clamshell portions are each pivotally supported from the handle between open and closed positions.
